(19) United States
(12) Patent Application Publication
(10) Pub. No.: US 2005/0214880 A1
(43) Pub. Date: Sep. 29, 2005

(54) Salivary soluble CD44: a molecular marker for head and neck cancer

(75) Inventors: Elizabeth J. Franzmann, Miami, FL (US); Vinata B. Lokeshwar, Miami, FL (US); Erika P. Reategui, Miami, FL (US)

Correspondence Address: VENABLE LLP, P.O. BOX 34385, WASHINGTON, DC, 20045-9998, US

(73) Assignee: University of Miami, Miami, FL (US)

(21) Appl. No.: 11/090,705

(22) Filed: Mar. 28, 2005

Related U.S. Application Data
(60) Provisional application No. 60/556,481, filed on Mar. 26, 2004.
(60) Provisional application No. 60/661,168, filed on Mar. 14, 2005.

(51) Int. Cl. C12Q 1/68; G01N 33/574

(52) U.S. Cl. 435/7.23; 435/6

(57) ABSTRACT

A method of diagnosing head and neck squamous cell carcinoma (HNSCC) includes obtaining a sample form a subject and measuring soluble CD44 (solCD44). The sample may be a body fluid such as saliva. An elevated level of solCD44 in the sample relative to baseline from a normal population of subjects is indicative of the presence of HNSCC. The degree of elevation can be indicative of the severity of disease. The HNSCC can be detected in an early stage and also as a recurrence of the disease.

The research resulting in this invention was supported by a grant from the National Institutes of Health, National Cancer Institute Grant number 5R03CA107828-02. The U.S. Government has certain rights in the invention.

### BACKGROUND OF THE INVENTION

1. Field of the Invention

The invention relates to a method of diagnosing head and neck squamous cell carcinoma in a subject by measuring soluble CD44 in a biological sample (e.g. saliva) obtained from the subject.

2. Background Information

Head and neck squamous cell carcinoma (HNSCC) accounts for approximately 90% of all head and neck malignancies (1). Unfortunately the majority of HNSCC patients present with advanced stage disease requiring multimodality therapy (2-5). Even with combinations of intensive chemotherapy, radiotherapy and surgery, cure rates reach only 40% for advanced stage disease (2, 5-7). Those cured often face serious morbidities including speech and swallowing problems, disfigurement and exorbitant healthcare costs. With nearly 40,000 incident cases per year, this disease poses a serious health concern in the United States (8).

HNSCC prognosis varies with site and stage of lesion at time of detection. In general, patients with laryngeal cancer have the best prognosis. Patients with hypopharyngeal cancer have the worst prognosis (9). HNSCC is staged I-IV based on the AJCC TNM (American Joint Committee on Cancer Tumor, Node, Metastasis) staging system (10). Tumor stage depends on site but generally T1-T3 indicates increasing tumor size and T4 indicates extension to adjacent sites. Node staging is based on size, number and side of neck involved and is uniform for all sites except the nasopharynx (6, 10). The risk of distant metastases (stage IV) increases with increasing neck disease (6). Cure is achieved in over 80% of stage I patients and over 60% of stage II patients. For patients with more advanced disease (stage III and IV), cure is attained in less than 30%.

Since five-year survival rates have not improved over several decades, and HNSCC is still rarely diagnosed in early stage when prognosis is best, early detection through screening is an important objective. To date, however screening for head and neck cancer by clinical exam has not proven effective (14). A clinical test should be simple and preferably noninvasive. Efforts are now focusing on molecular approaches to early detection, using either nucleic-acid based tools or protein-based tools. Although there still is a possibility that serum will yield a useful early-stage marker for HNSCC, saliva is a preferable patient fluid. Saliva has an advantage over blood in that there is easy access and it does not require invasive collection (81). Moreover, saliva is in direct contact with the tissue from which the HNSCC derives. The average daily production of whole saliva varies between 1 and 1.5 liters. The majority (65%) of saliva in the unstimulated state originates from submandibular glands, 20% from the parotid gland and the remainder from sublingual and minor salivary glands located throughout the upper aerodigestive tract (UADT). Fifty percent of flow in the stimulated state derives from the parotid glands (82). Ninety-nine percent water, saliva contains a variety of electrolytes, immunoglobulins, proteins, enzymes, mucins, and nitrogenous products and is hypotonic especially in the unstimulated state (82). Normal pH ranges from 6-7. While salivary protein content can be affected by circadian variations, stress and other factors, in general it increases proportionally with increasing flow rate (82). The salivary flow rate is influenced by the size of the salivary glands, hydration status, nutritional state, stimulus, and gender (83). Total protein concentrations of whole saliva in the unstimulated state give an accurate indication of the hydration state of an individual (84). So the saliva may provide simple noninvasive access to tissue throughout the UADT.

Hu and Sidransky recently reviewed many of the nucleic acid-based tools for head and neck cancer screening (63, 64). Spafford et. al. showed either loss of heterozygosity or microsatellite instability in one of 23 markers saliva samples from HNSCC patients and in none healthy control subjects (15). Microsatellite analysis holds promise (15), but is somewhat costly (16, 17). Boyle et. al. identified tumor-specific p53 genetic mutations in saliva samples from 5 of 7 patients with HNSCC (65). Abnormal promoter hypermethylation has been investigated as a marker in saliva HNSCC patients (66). Mitochondrial DNA mutations have been detected in 6 of 9 saliva samples from HNSCC by direct sequencing (67). More recently quantitative PCR analysis of mRNA in saliva showed that a panel of 4 markers detects HNSCC with 91% sensitivity and 91% sensitivity (75). Such feasibility studies show promise, but none of these markers have been validated in large trials.

Protein-based early detection tools have the advantage over nucleic-acid based techniques in that they evaluate the end product of translation and can detect post-transcriptional and post-translational changes that may take place as a result of tumorigenesis. In analyzing proteins, one can study either protein activity or protein levels or sites of expression. Some protein activity studies include salivary hyaluronic acid (HA), salivary hyaluronidase (HAase) and Interleukin-8, and telomerase (28, 31, 76). Telemerase activity was studied using a PCR-based assay. Activity was found in 80% of HNSCC patients and 5% of normals (76).

The ELISA system is the most well-established, sensitive and widely available protein-based testing platform for the detection of cancer markers in body fluids or tissue (121). Several markers have been studied using ELISA or ELISA-like assays. As with the already-described tests, protein-based studies have shown feasibility, but none have not been validated in large trials. A marker is needed that is robust in clinical tests.

Another candidate molecular marker for early stage HNSCC is CD44. CD44 comprises a family of isoforms and variants are expressed in many cell types (20-24). CD44 isoforms mediate a direct link between the extracellular matrix and the cytoskeleton via their conserved extracellular HA binding regions and intracellular ankyrin binding regions (20, 29). CD44 proteins are also released in soluble form (solCD44) via proteases (30) and are detectable in normal circulation (26, 31-36). These isoforms arise from alternative splicing of a variable exon region present in CD44 mRNA (25). They differ in primary amino acid sequence as well as in amount of N— and O-glycosylation (85,86). Isoforms such as CD44 standard (CD44s), CD44 epithelial (CD44E or CD44v8-10) (25), and CD44v3-v10 in keratinocytes (26) exist in normal cells. Other CD44 variant isoforms (CD44v) are differentially expressed in some tumors (27,28). The standard form contains only the common domain.

CD44s and many variants are upregulated in many tumors (27, 28 37-40). Circulating levels of solCD44 correlate with metastases in some tumors (31-34). Concentrations of solCD44v5 and solCD44v6 in serum of smokers are dose related but reversible with smoking cessation, while total solCD44 isoform concentrations do not change with smoking status (35). When plasma levels of solCD44v6 were measured in HNSCC patients and controls no significant difference was seen (36). In addition, most circulating CD44v6 proteins were derived from normal epithelial compartments, including breast cells, colon cells and squamous cells (36). For HNSCC, a comprehensive study of the utility of CD44 markers has not been done.

Immunohistochemical analyses and ELISA have investigated CD44 as a prognostic indicator for HNSCC, however, the reports are conflicting (79, 87). Evidence suggests that CD44 isoforms are upregulated in dysplastic lesions compared to normal tissue (77, 78). Concentrations of solCD44v5 and solCD44v6 in smokers are dose related and reversible with smoking cessation, while total solCD44 isoform concentrations do not change with smoking status (35). When plasma levels of solCD44v6 were measured in HNSCC patients and controls, no significant difference was seen (36). Similarly, when total serum solCD44 levels were measured in another study, they tended to correlate with tissue levels (which were increased) but this tendency did not reach statistical significance (79). Conversely, our pilot study on saliva specimens from 26 HNSCC patients and 10 normal controls indicated that solCD44 was elevated in almost 80% of the HNSCC patients with mucosally invasive disease but in none of the controls.

Since patients are far more likely to be cured at first treatment rather than after recurrence (80), (56), both early detection and predictive markers for HNSCC are needed.

### SUMMARY OF THE INVENTION

A clinically useful test should be simple and noninvasive. Saliva has an advantage over blood in that there is easy access and it does not require invasive collection (81). As noted above, the average daily production of whole saliva is significant (between 1 and 1.5 liters) and access to cells from the UADT through saliva collection is simple and noninvasive.

CD44 has been investigated as both a prognostic marker and an early detection tool for HNSCC. Since solCD44 is detectable at high levels in conditioned media of HNSCC cell lines, it is likely that the source of solCD44 in saliva from HNSCC patients is, at least in part, from tumor cells.

It is one object of the invention to provide a method of diagnosing head and/or neck squamous cell carcinoma (HNSCC) in a subject, comprising measuring soluble CD44 (solCD44) in the subject. It has been found that an elevated level of solCD44 in the subject compared to a baseline from a normal population of subjects is indicative of the possible presence of HNSCC.

It is also an object of the invention to provide a method of diagnosing head and/or neck squamous cell carcinoma (HNSCC) in a subject, comprising measuring soluble CD44 (solCD44) in the subject over time, compare the level of solCD44 to a baseline from the same individual established in an earlier measured level and assess the possible presence of HNSCC.

The meaning of “head and neck squamous cell carcinoma” will be clear to those of skill in the art and means squamous cell carcinomas of the head and neck region including but not limited to mouth (e.g. floor of the mouth, the tongue, soft palate, hard palate, anterior tonsillar pillar, and the retromolar trigone), pharynx and larynx.

The word “subject” is used generally to indicate any mammal that may be at risk of, or suffering from, HNSCC. In certain contexts, the term may also include subjects who are asymptomatic but may be at high risk based on genetic or environmental factors. In certain contexts, the term may also include normal subjects, who are believed to be free of disease and at low risk, and who may be tested to provide baseline values for screening. Generally, the term subject indicates a human.

It is another object to provide a method of predicting the course of HNSCC in a subject, comprising measuring solCD44 in the subject, wherein the degree of elevation of the level of solCD44 in the subject compared to a baseline from a normal population of subjects or a baseline of the same asymptomatic individual is indicative of the severity of disease.

By “same asymptomatic individual” is meant the individual at a time when he/she is known or believed to have been free of disease.

It is yet another object to provide a method of predicting the success of treatment of HNSCC in a subject, comprising measuring solCD44 in the subject, wherein the degree of elevation of the level of solCD44 in the subject compared to a baseline from a normal population of subjects or a baseline of the same asymptomatic individual is predictive of outcome, with a low elevation level being more favorable than a high elevation level.

It is yet another object to provide a method of determining the effectiveness of treatment of HNSCC in a subject, comprising measuring solCD44 in the subject, wherein a decrease in the level of solCD44 in the subject compared to prior levels measured in the subject is indicative of effective treatment.

It is yet another object to provide a method of monitoring recurrence of HNSCC in a subject, comprising measuring solCD44 in the subject, wherein an increase in the level of solCD44 in the subject compared to prior levels measured in the subject is indicative of recurrence.

It is yet another object to provide a method of monitoring recurrence of HNSCC in a subject, comprising measuring solCD44 in the subject, wherein an increase in the level of solCD44 in the subject compared to baseline of a population with a history of HNSCC is indicative of recurrence.

By “biological sample” is meant a tissue sample or a sample of a bodily fluid from said subject. By “bodily fluid” is meant, for example, blood, urine, perspiration, and in particular, saliva. In the methods described herein, solCD44 is preferably measured in a bodily fluid, for example, in saliva.

In one embodiment of the method of the invention, HNSCC is measured in early stage. Head and neck cancer is staged I-IV based on the AJCC TNM staging system (10). Tumor stage depends on site but generally T1-T3 indicates increasing tumor size and T4 indicates extension to adjacent sites. Node staging is based on size, number and side of neck involved and is uniform for all sites except the nasopharynx (6, 10). By “early stage HNSCC” is meant stage I or Stage II, according to these criteria. The ability to diagnose HNSCC at an early stage, provide a prognosis, and measure treatment efficacy is particularly advantageous when overt physical symptoms may be difficult to detect or measure. Stage I and II are generally considered early disease while III and IV are generally considered late stage. While an early detection test has greatest benefit if it detects a disease in stage I rather than Stage IV, even early stage IV e.g., no distant metastases, is better than late stage IV (with metastases).

It is yet another object to provide a method of diagnosing HNSCC in a subject, comprising measuring solCD44 in saliva of said subject, wherein an elevated level of solCD44 in the subject compared to a baseline of solCD44 in saliva of a normal population of subjects is indicative of the possible presence of HNSCC.

Measurement of solCD44 may be made in any tissue sample or bodily fluid, but is particularly convenient in saliva. In one embodiment a quantity of normal saline (e.g. 5 ml) is placed in a subject's mouth, and the subject is asked to rinse (swish and/or gargle) prior to spitting into a specimen cup. Specific instructions to the subject may vary, according to the experience and knowledge of the practitioner conducting the test, but in general more reliable results will be obtained if the same “rinsing” procedure is followed by each subject. For example, the subject may be asked to swish for five seconds, gargle for five seconds and then spit into a specimen cup. This process increases the amount of UADT mucosa that contacts the sample. Preferably, samples are preserved by adding a proteinase inhibitor in suitable amounts, cooling to ice bath temperatures and storing at −80° C. Levels of solCD44 may be measured by any means known to those of skill in the art, for example, using an ELISA assay (Bender MedSystems, Vienna Austria) that recognizes all solCD44 normal and variant isoforms (total solCD44).

Soluble CD44 can be measured in normal controls, HNSCC cell lines and a CD44 negative cell lines as well as patients to provide baseline values with which to compare values of the patients. Mean solCD44 levels for each sample (control, patient or cell line) are divided by the average protein concentration for that sample. The normalized solC44 levels are then averaged and standard deviation determined. Because the protein concentration in tumor samples were significantly elevated compared to normals we determine both the normalized solCD44 and solCD44 for all samples. If either level is elevated compared to normals, the test is considered positive. Persons of skill in the art will be able to determine using routine experimentation the threshold level for a positive determination in specific circumstances.

### BRIEF DESCRIPTION OF THE DRAWING

FIG. 1. Western blot analysis of cell line conditioned media, HNSCC participants and normal control. The FaDU and SCC-11B cell lines show major bands ranging from approximately 70-75 kDa corresponding to the soluble form of CD44 described in the literature (30). The COS-7 cell line, which is CD44 negative, does not show the 65-70 kDa band. The 3 HNSCC patients show major bands ranging from approximately 65-75 kDa, that correspond to the CD44 protein, in the two normal controls this band is faint or undetectable.

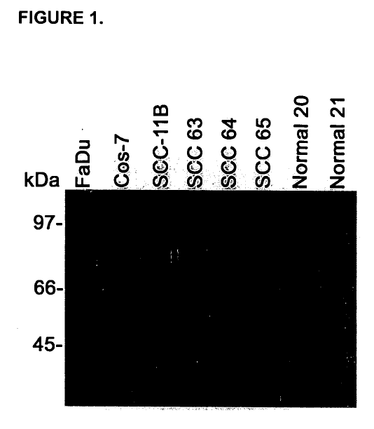

FIG. 2. ROC Curve for SolCD44 in 33 early stage HNSCC patients.

FIG. 3. Representative standard curve for solCD44 ELISA.

FIG. 4. Western blot analysis of cell line conditioned media (FIG. 4A) and saliva from HNSCC participants and normal controls (FIG. 4B). All the samples from HNSCC cell lines and patients show bands in the 65-70 kDa range as expected for the soluble form of CD44 standard (21,32). Additional bands seen at 40 kDa and 50 kDa have also been described (32). Since isoforms other than CD44 standard are present in HNSCC, the 30 kDa band is likely a result of proteinase-mediated cleavage of additional isoforms rather than problems with antibody specificity. This is further supported by our normal samples and the Cos-7 cell lines, which show no bands or a faint band in the 65-68 kDa region (Normal 20).

### DETAILED DESCRIPTION OF THE INVENTION

### EXAMPLE 1

We performed a solCD44 ELISA on saliva from 26 HNSCC patients, 10 normal volunteers, conditioned media (CM) of 4 HNSCC cell lines and 1 CD44 negative cell line (COS-7). Western blot was performed on CM from 2 HNSCC cell lines (UMSS11B and FaDu), COS-7, 3 HNSCC and 2 normal saliva specimens to verify ELISA antibody specificity.

The solCD44 ELISA was performed on HNSCC cell lines to verify that solCD44 is expressed by the cancer cells.

We also performed western blot analysis on HNSCC cell lines and saliva from HNSCC patients and normal volunteers to confirm the specificity of the solCD44 ELISA antibody.

### Materials and Methods

Subject Characteristics

Twenty-six HNSCC patients and 10 normal controls were obtained according to the protocol approved by the Institutional Review Board. Control subjects were volunteers from healthcare and research fields. To decrease the potential for false negatives in our control group, all were nonsmokers for at least 10 years. All subjects completed a written consent prior to enrollment. Patient characteristics are shown in Table 1. These included four patients who denied history of smoking or drinking. All patients had biopsy proven newly diagnosed or recurrent squamous cell carcinoma. We included all stages and sites except nasopharynx, since nasopharyngeal carcinoma tends to behave differently than squamous cell carcinoma in other sites.

TABLE 1
Patient Characteristics

| | | Tumor | | | | | | Tobacco or |
|---|---|---|---|---|---|---|---|---|
| Patient | Site | size* | Node* | Metastases* | Stage* | Recurrence | Radiation | alcohol |
| 13 | larynx | 4 | 0 | 0 | IV | no | no | yes |
| 14 | oropharynx | 2 | 0 | 0 | II | no | no | no† |
| 15 | oral cavity | 4 | 0 | 0 | IV | yes | yes | yes |
| 16 | oral cavity | 2 | 2 | 0 | IV | yes | no | no† |
| 18 | larynx | 1 | 0 | 0 | I | no | no | yes |
| 19 | hypopharynx | 4 | 0 | 0 | IV | yes | yes | yes |
| 20 | oral cavity | 3 | 1 | 0 | III | no | no | yes |
| 23 | hypopharynx | 4 | 0 | 0 | IV | no | no | yes |
| 24 | larynx | 1 | 0 | 0 | I | no | no | yes |
| 25 | larynx | 4 | 1 | 1 | IV | yes | yes | yes |
| 26 | lymph node | 0 | 1 | 0 | III | yes | no | yes |
| | metastasis | | | | | | | |
| 27 | oral cavity | 2 | 0 | 0 | II | yes | yes | yes |
| 28 | larynx | 1 | 0 | 0 | I | no | no | yes |
| 29 | hypopharynx | 3 | 2 | 0 | IV | no | no | yes |
| 30 | hypopharynx | 4 | 2 | 0 | IV | no | no | yes |
| 31 | oral cavity | 1 | 0 | 0 | I | no | yes** | yes |
| 32 | larynx | 0 | 0 | 0 | 0 | no | no | yes |
| 33 | oropharynx | 1 | 0 | 0 | I | no | no | yes |
| 34 | oral cavity | 4 | 0 | 0 | IV | no | no | yes |
| 35 | oral cavity | 4 | 2 | 0 | IV | no | no | yes |
| 36 | hypopharynx | 4 | 0 | 0 | IV | no | yes** | yes |
| | larynx | 1 | | | | | | |
| 37 | larynx | 4 | 2 | 0 | IV | no | no | yes |
| 38 | larynx | 4 | 0 | 0 | IV | no | no | yes |
| 39 | larynx | 4 | 0 | 0 | IV | no | no | yes |
| 40 | oropharynx | 2 | 2 | 0 | IV | yes | yes | no |
| 42 | oropharynx | 1 | 1 | 1 | IV | no | no | No |

*Tumor, node, metastasis and stage based on AJCC criteria (10).
**This is a second primary for the patient.
†Social alcohol use only

Saliva Collection

Five milliliters of normal saline was placed in the subject's mouths. Patients were asked to swish for five seconds, gargle for five seconds and then spit into a specimen cup. Saliva was placed on ice for transport and stored at −80° C.

Cell Culture

FaDu (hypopharyngeal carcinoma) and COS-7 (CD44 negative cell line) were obtained from the American Type Culture Collection. MDA-1483 (oral cavity cancer) was a gift from Dr. Mien-Chie Hung, University of Texas M.D. Anderson Cancer Center. UM-SCC-9 (tonsil SCC) and UM-SCC11B (hypopharynx SCC) were gifts from Dr. T E Carey, University of Michigan FaDu, COS-7, and MDA-1483 were grown in RPMI medium. UMSCC-9 and UMSCC-11B were grown in DMEM (Dulbecco's Modified Eagle's Medium). All cell line media were supplemented with 10% fetal bovine serum, streptomycin and penicillin. At approximately 60% confluence, cultures were washed and incubated in serum free media supplemented with insulin, transferrin and selenium. These conditioned media (CM) were collected at 48-72 hours.

By testing the fluid in which these cell lines grew we were able to show that these cells produce solCD44. This provides evidence that solCD44 in saliva comes at least in part from squamous carcinoma cells (as expected).

Salivary SolCD44 ELISA

We measured levels of solCD44s using an ELISA assay (Bender MedSystems, Vienna, Austria) that recognizes all solCD44 normal and variant isoforms (total solCD44). The principles of the test involve a sandwich-type ELISA where a monoclonal anti-solCD44 antibody, adsorbed onto microwells, binds CD44 in the sample. Horseradish peroxidase-conjugated monoclonal anti-solCD44 antibody binds the CD44-antibody complex and reacts with a substrate solution to produce a colored product with an absorbance measured quantitatively at 450 nm. Sample concentrations are determined by a standard curve.

The saliva samples described above are vortexed, centrifuged at 3,000 G and the supernatant is used for study. The manufacturer's protocol can be followed with slight modifications. For example, in the pilot study of the invention, rather than the recommended dilution of 1:60, the test was performed at full and half concentration for each sample. Other suitable dilutions can be determined by those of skill in the art using routine experimentation. To correct for varying concentrations of the saliva sample, solCD44 level is normalized to protein content (for example, as described by Lokeshwar (17,36-38)), using any standard protein assay. For example, the assay marketed by Bio-Rad (Hercules, Calif.) may be used according to manufacturer's protocol with saliva samples at full and half concentration. All samples were performed in triplicate.

Statistical Analysis

Characteristics and solCD44 results for both patients and normal volunteers were entered into a computer database. Statistical analyses were performed using programs of the SAS Institute, Inc (Version 8.2). The protein and solCD44 concentrations for each sample were averaged and standard deviation calculated. The triplicate solCD44 levels for each sample were divided by the average protein concentration for that sample. The normalized solCD44 levels were then averaged and standard deviation determined. We compared solCD44 and normalized solCD44 levels between normal volunteers and cancer patients and between specific subgroups of cancer patients based on characteristics such as stage, site and tumor size. For comparison of two groups, Student's t-test was used. Analysis of variance was used to compare solCD44 levels when more than two groups were being compared.

Western Blot Analysis

To verify the specificity of the anti-solCD44 antibody used in the ELISA test, a standard western blotting technique was performed on CM from 4 HNSCC cell lines (UMSCC9, MDA-1483, UMSS11B and FaDu) a CD44 negative cell line COS-7, 3 HNSCC and 2 normal saliva specimens. To avoid degradation, we added proteinase inhibitors to fresh saliva specimens (normal and tumor). All samples were vortexed, centrifuged at 700 rpm for 5 minutes, normalized for protein content and subjected to electrophoresis on a 12% SDS-polyacrylamide gel under reducing conditions. Proteins were transblotted onto nitrocellulose membranes (Protran, pure nitrocellulose transfer and immobilization membrane, BioScience). The blot was washed with TTBS (20 mM tris, 500 mM sodium chloride, 0.1% tween-20) followed by blocking with 5% milk. Primary antibody, anti-solCD44, (anti-CD44s by Bender Medsystems, Vienna, Austria), was incubated with the membrane overnight at a concentration of 1:3000 with 5% milk in TTBS. After washing with TTBS, the secondary antibody (anti-mouse IgG, biotin conjugate) was applied diluted 1:1000 with TTBS and incubated 1 hour followed by washing. The membrane was treated with streptavidin-biotinylated alkaline phosphatase complex (Amplified Alkaline Phosphatase Immun-blot Assay Kit, Bio-Rad Laboratories) for 2 hours with gentle agitation at room temperature followed by washing. Protein bands were visualized using a color development solution (AP Conjugate Substrate Kit, Bio-Rad). The membrane was allowed to dry, scanned and stored via Adobe Photoshop 7.0 software.

### Results

ELISA

Mean values for the solCD44 test were 7.85 ng/ml for HNSCC patients and 1.09 ng/ml for normals (p<0.001). Mean values for normalized solCD44 test were 10.76 ng/mg protein for HNSCC patients and 1.80 ng/mg protein for normals (p<0.001). SolCD44 levels did not vary significantly with stage, recurrence, history of radiation treatment, tobacco and alcohol risk factors, site or size (Table 2). Results for normal volunteers are listed in Table 3. Results for HNSCC patients are listed in Table 4 by tumor site.

TABLE 2
SolCD44 levels of 26 samples from cancer patients by
clinical characteristics

| Cancer vs normal | N (%) | Mean | SD | P value |
|---|---|---|---|---|
| Cancer | 26 | 7.85 | 6.33 | <0.001* |
| Normal | 10 | 1.09 | 0.69 | |
| Clinical | | | | 0.96* |
| Characteristics | | | | |
| Stage | | | | |
| I, II, III | 10 (38) | 7.94 | 3.76 | |
| IV | 16 (62) | 7.80 | 7.04 | |
| Recurrence | | | | 0.52* |
| Yes | 7 (27) | 10.50 | 8.81 | |
| No | 19 (73) | 6.88 | 3.74 | |
| Radiation | | | | 0.29* |
| Yes | 7 (27) | 12.11 | 13.13 | |
| No | 19 (73) | 6.28 | 4.84 | |
| Tumor Site (n = 25) | | | | 0.07** |
| Oral cavity | 7 (28) | 8.08 | 6.31 | |
| Larynx | 9 (36) | 4.78 | 3.55 | |
| Oropharynx | 4 (16) | 16.72 | 15.91 | |
| Hypopharynx | 5 (20) | 4.73 | 3.32 | |
| Tumor size | | | | 0.56** |
| 0 | 2 (8) | 8.02 | 8.54 | |
| 1 | 6 (23) | 7.48 | 5.31 | |
| 2 | 4 (15) | 14.28 | 17.85 | |
| 3 | 2 (8) | 6.99 | 7.66 | |
| 4 | 12 (46) | 6.02 | 4.19 | |
| Risk factors | | | | 0.34 |
| Positive | 22 (85) | 6.39 | 4.75 | |
| Negative | 4 (15) | 15.89 | 16.85 | |

*t-test.
**ANOVA

TABLE 3
Salivary solCD44 levels of normal volunteers and determination of
preliminary cut-off point

| Normal | Sol CD44 ± SD | Ng Sol CD44/mg protein ± SD |
|---|---|---|
| 1 | 0.00 | 0.00 |
| 2 | 1.08 ± 0.09 | 2.22 ± 0.18 |
| 3 | 0.23 ± 0.40 | 0.69 ± 1.20 |
| 4 | 1.87 ± 0.11 | 2.26 ± 0.14 |
| 5 | 0.24 ± 0.42 | 1.05 ± 1.81 |
| 6 | *1.74 ± 0.96 2.7 | 1.58 ± 0.87 |
| 7 | 1.19 ± 0.12 | 1.15 ± 0.11 |
| 8 | 1.58 ± 0.30 | **3.64 ± 0.69 4.3 |
| 9 | 1.21 ± 0.03 | 3.51 ± 0.08 |
| 10 | 1.73 ± 0.61 | 1.86 ± 0.66 |

*Highest normal value for solCD44 test. The cut-off point is 2.7 which includes error associated with triplicate measurements.
**Highest normal value for normalized solCD44 test. The cut-off point is 4.3 which includes the error associated with triplicate measurements.

TABLE 4
Results of SolCD44 ELISA in HNSCC patients

| | | | | |
|---|---|---|---|---|
| | | Ng SolCD44 ∀ | Ng SolCD44/ | |
| Patient | Tumor size | SD | mgprotein ∀SD | Result |

Oral Cavity

| | | | | |
|---|---|---|---|---|
| 15 | 4 | 2.83 ± 0.41 | 6.53 ± 0.94 | + |
| 16 | 2 | 0.85 ± 0.04 | 1.29 ± 0.05 | − |
| 20 | 3 | 12.41 ± 1.54 | 18.82 ± 2.33 | + |
| 27 | 2 | 3.76 ± 0.95 | 37.73 ± 9.53 | + |
| 31 | 1 | 16.54 ± 1.53 | 8.44 ± 0.78 | + |
| 34 | 4 | 5.52 ± 0.61 | 5.17 ± 0.57 | + |
| 35 | 4 | 14.67 ± 2.26 | 9.77 ± 1.51 | + |

Laryngeal

| | | | | |
|---|---|---|---|---|
| 13 | 4 | 11.81 ± 3.84 | 16.88 ± 5.49 | + |
| 18 | 1 | 3.8 ± 1.11 | 14.85 ± 4.28 | + |
| 24 | 1 | 2.22 ± 0.21 | 2.10 ± 0.20 | − |
| 25 | 4 | 6.60 ± 0.73 | 10.23 ± 1.12 | + |
| 28 | 1 | 7.86 ± 0.30 | 5.46 ± 0.21 | + |
| 37 | 4 | 2.48 ± 0.82 | 7.20 ± 2.37 | + |
| 38 | 4 | 5.61 ± 1.06 | 11.15 ± 2.11 | + |
| 39 | 4 | 0.64 ± 0.60 | 1.49 ± 1.38 | − |

Oropharyngeal

| | | | | |
|---|---|---|---|---|
| 14 | 2 | 12.52 ± 0.70 | 8.21 ± 0.46 | + |
| 33 | 1 | 4.20 ± 0.25 | 3.38 ± 0.20 | + |
| 40 | 2 | 40.0 | 28.45 | + |
| 42 | 1 | 10.17 ± 1.31 | 35.60 ± 4.60 | + |

Hypopharyngeal

| | | | | |
|---|---|---|---|---|
| 19 | 4 | 5.38 ± 0.22 | 11.43 ± 0.47 | + |
| 23 | 4 | 5.18 ± 1.13 | 3.33 ± 0.73 | + |
| 29 | 3 | 1.58 ± 0.41 | 1.80 ± 0.46 | − |
| 30 | 4 | 1.78 ± 0.40 | 1.63 ± 0.36 | − |
| 36 | 4 | 9.72 ± 0.99 | 22.81 ± 2.33 | + |

No Mucosal Invasion

| | | | | |
|---|---|---|---|---|
| 32 | 0 | 1.98 ± 0.28 | 2.46 ± 0.35 | − |
| 26 | 0 | 14.05 ± 1.99 | 3.64 ± 0.52 | + |

TABLE 5
Results of solCD44 ELISA in cell lines

| Cell line | Ng SolCD44 | Ng SolCD44/mg protein |
|---|---|---|
| SCC11B | 17.51 | 11.36 |
| SCC9 | 28.15 | 20.61 |
| FaDu | 23.02 | 22.18 |
| MDA-1483 | 17.17 | 9.90 |
| COS-7 | 0 | 0 |

The mean protein concentration was significantly higher in the saliva from the subjects with tumors than the normal controls (0.97 mg versus 0.61 mg p=0.05). This result contradicts results of other studies (19, 46). However, in a study conducted in India (46), staging was not addressed and most of the tumors were located in the buccal mucosa which may obstruct secretions from the parotid duct. Buccal mucosa is a common lesion site associated with the Indian practice of areca nut chewing, but an uncommon site for HNSCC in the United States. Since we had more HNSCC patients in our study than the other two studies, it is possible that protein concentration in HNSCC may be elevated compared to normal controls. Specific proteins (HAase) were elevated in saliva of tumor patients compared to controls in another study (19). If protein concentrations are higher in HNSCC patients compared to normal controls for reasons other than hydration, normalized solCD44 levels in HNSCC may be falsely lowered. For this reason, we examined both solCD44 concentrations and normalized solCD44 concentrations. 10 of 10 normal saliva samples had solCD44 levels below 2.7 ng and normalized solCD44 levels below 4.3 ng/mg protein. To analyze our preliminary data further, we considered a HNSCC detected if the saliva specimen had solCD44 above 2.7 ng or normalized solCD44 levels above 4.3 ng/mg protein. With this assumption, the test detected 77% of all HNSCC samples studied and 79% of invasive HNSCC. By site the test detected 10/11 oral cavity and oropharynx cancers, 9/13 hypopharynx and larynx cancers, and 1/2 HNSCC without mucosal invasion.

SolCD44 ELISA tests performed on cell lines showed that CD44 was secreted by the HNSCC cells (see Table 5). This suggests that solCD44 in the saliva is secreted, at least in part, by epithelial cells. The CD44 negative COS-7 cell line did not show any solCD44 as expected.

Western Blot Analysis

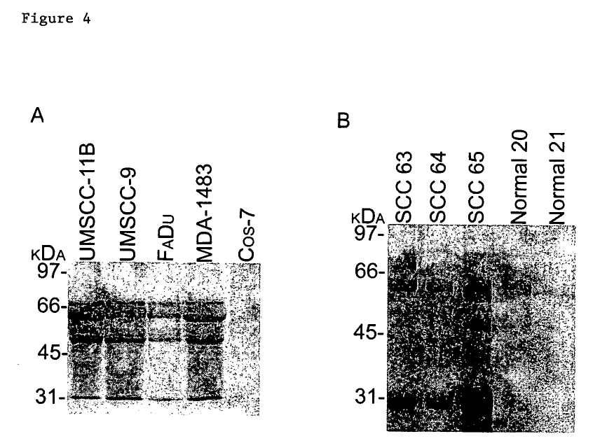

Results of western blot reveal several bands in saliva of HNSCC patients and CM of HNSCC cell lines (FIGS. 4A and 4B). This banding pattern is explained by proteinase-mediated cleavage of CD44 as described by Nakamura et al⁸⁹. In their study, the CD44 negative A375 cell line was transfected with CD44 standard form. MT1-MMP-mediated cleavage resulted in three heterogeneously glycosylated fragments with bands of 65-70 kDa, 50-60kDa, and 37-40 kDa. Our western blots show bands at 65 and 68 kDa for cell line CM and HNSCC saliva, which is within the range described by Nakamura et al. The HNSCC saliva sample SCC65 shows 40 and 50 kDa bands and all the cell lines show the 50 kDa band, also consistent with the Nakamura et al findings. HNSCC is known to express multiple CD44 isoforms. The 30 kDa band is likely a result of proteinase-mediated cleavage of additional isoforms rather than problems with antibody specificity. This is further supported by our normal samples and the Cos-7 cell lines, which show no bands or a faint band in the 65-70 kDa region (Normal 20).

### EXAMPLE 2

The Bender MedSystems ELISA plate is designed for use with plasma, serum and urine samples. Any matrix (e.g. serum, urine, saliva) may contain factors that affect ELISA test results, commonly known as a matrix effect. Such effects can be corrected for by running the standards in the same matrix as the samples. To better adapt the test to saliva specimens, we prepared our standards in a synthetic saliva matrix (Salimetrics, State College, Pa.) diluted 1:5 in normal saline (since patients swish and gargle with 5 cc saline) and switched to a sample diluent (Salimetrics) developed for saliva samples.

We performed the solCD44 ELISA on 73 HNSCC patients and 54 controls with other head and neck diseases. This includes the 25 invasive HNSCC saliva samples from Example 1, which were retested using the revised method. Sixty-nine of the HNSCC patients had mucosal disease of the oral cavity, oropharynx, larynx or hypoparynx. Results are summarized in Table 6. The mean solCD44 level was 27.6 ng/ml for the HNSCC with mucosal disease and 7.36 ng/ml for the controls (p<0.0001). Levels tended to be higher in patients with oral cavity and oropharynx tumors compared to larynx and hypopharynx tumors. Levels did not correlate significantly with tumor stage, history of previous HNSCC (recurrence or second primary) or history of prior radiation. An additional 4 HNSCC patients with cervical lymph node disease but no identified mucosal primary had significantly elevated mean salivary solCD44 levels compared to the control group (22.0 ng/ml, p<0.0001) suggesting that the salivary solCD44 test is able to detect clinically occult mucosal disease.

TABLE 6
Means for Salivary SolCD44 Level

| | | | Mean | | |
|---|---|---|---|---|---|
| | | N | (ng/ml) | Std Dev | p value |
| Combined | Cancer | 69 | 27.622 | 37.199 | <.0001 |
| Group | Normal | 54 | 7.360 | 5.955 | |
| Tumor Site | Larynx/ | 27 | 14.878 | 10.806 | 0.0060 |
| | Hypopharynx | | | | |
| | Oral Cavity/ | 42 | 35.815 | 45.225 | |
| | Oropharynx | | | | |
| Tumor Stage | I-II | 31 | 22.599 | 26.697 | 0.2921 |
| | III-IV | 38 | 31.720 | 43.896 | |
| Recurrence | No | 57 | 26.965 | 38.040 | 0.7518 |
| Status | recurrence | | | | |
| | Recurrence | 12 | 30.743 | 34.276 | |
| 2nd Primary | 2nd Primary | 7 | 27.960 | 31.271 | 0.9800 |
| | No 2nd | 62 | 27.584 | 38.031 | |
| | Primary | | | | |
| Radiation | No RT | 54 | 25.939 | 37.869 | 0.4798 |
| Therapy (RT) | RT received | 15 | 33.682 | 35.241 | |

Ideally, a population-based screening test for HNSCC would detect disease at an early treatable state. For this reason we further analyzed a subset of HNSCC patients with early disease and no prior history of head and neck cancer. We included stage III disease in this analysis to avoid selecting for slowly growing, nonaggressive tumors. Thirty-three patients met these criteria. The mean solCD44 level for this group was significantly elevated compared to the control group (26.5 ng/ml, p<0.005).

A subset of patients completed a questionnaire containing information on potentially important covariates including tobacco and alcohol exposure, race, ethnicity, gender, and SES. In addition, they received an oral examination and assessment of their ability to gargle. We have this information available on 18 stage I-III newly diagnosed HNSCC and 48 normal controls. We further studied solCD44 expression in this group. The level of expression of solCD44 was also statistically significantly elevated in this cancer group compared to normal group (23.9 ng/ml vs 7.0 ng/ml, p<0.05). The distribution of potentially important covariates was compared between the two groups by Chi-square analysis. The groups differed significantly with respect to several factors. Compared to the control patients, cancer patients were older, more likely male, less educated, reported less income, were more likely to have ever smoked cigarettes (>100 cigarettes in a lifetime), and were more likely to have poor oral health. Multiple regression analysis was used to adjust for these factors. Despite the imbalance in these characteristics, the level of expression of solCD44 remained statistically significantly elevated this subset of cancer patients compared to controls after adjustment.

Since salivary protein levels correlate with hydration status, protein assays were performed as previously described to correct for hydration status. Protein levels were significantly higher in the 69 cancer patients (1.05 mg/ml, p<0.0001) and 33 stage I-III patients (1.07 mg/ml, p<0.0001) compared to controls (0.46 mg/ml). Protein levels were significantly higher in oral cavity/oropharynx (1.22 mg/ml) than larynx/hypopharynx (mean 0.79 mg/ml, p<0.01) but did not vary with tumor stage, prior history of HNSCC or history of previous radiation.

Despite generally increased protein levels in HNSCC patients, the mean normalized solCD44 levels were significantly higher in the 69 HNSCC patients (32.8 ng/ml) compared to controls (18.7 ng/ml, p<0.05). Normalized solCD44 levels did not correlate with tumor site, stage, recurrence, second primary status or history of prior radiation. Differences in normalized salivary solCD44 means between the 33 stage I-III patients (35.7 ng/ml) and controls did not reach statistical significance (p=0.17). These results suggest that normalized levels have the potential to detect elevated CD44 levels in relatively overhydrated patients that would otherwise not be detected with unnormalized solCD44. However, because protein levels are increased in tumor patients compared to controls, solCD44 level alone must also be analyzed. Since normalized solCD44 did not reach statistical significance in the stage I-III target group, further discussion will focus on the unnormalized solCD44 test.

Cut-Off Point, Sensitivity and Specificity

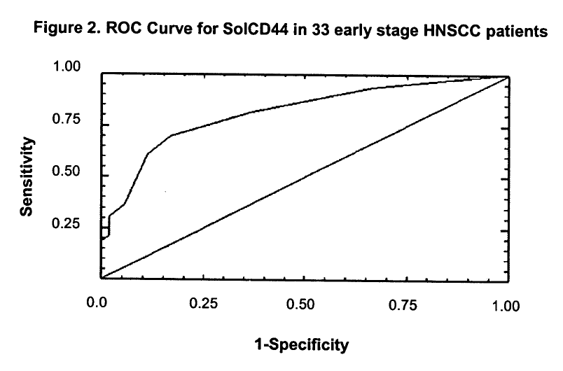

Using results from our target group of 33 newly diagnosed stage I-III HNSCC and 54 controls with benign diseases of the UADT, the sensitivity and specificity of the solCD44 test was calculated at several cut-off points, thereby deriving its receiver-operator characteristic (ROC) curve. The ROC curve is shown in FIG. 2. A cut-off point set at 11.3 ng/ml resulted in a sensitivity of 70% and specificity of 83%. Our control group was designed to specifically investigate other common UADT diseases that may confound results and adversely affect specificity. Even with this, results are comparable to other widely used screening tests such as prostate specific antigen for prostate cancer (sensitivity 60-80%, specificity 90%)(71) and the Papanicolaou test for cervical cancer (sensitivity 30-87%, specificity 86-100%) (72).

We also obtained information on the effectiveness of gargle on 17 cancer patients and 48 controls. Two of the eighteen cancer patients and 11 of 48 controls had some limitation in their ability to gargle as scored on a scale from 0-2 with 2 being a good gargle. Both of the cancer patients with limited gargle (score 0 or 1) had false negative results. The tumor patients and controls did not differ in ability to gargle by Chi-square analysis (p=0.32). The differences in mean solCD44 level between controls who were good garglers (7.65 ng/ml) and limited garglers (4.85 ng/ml) nearly meets statistical significance (p=0.051).

Standard Curve

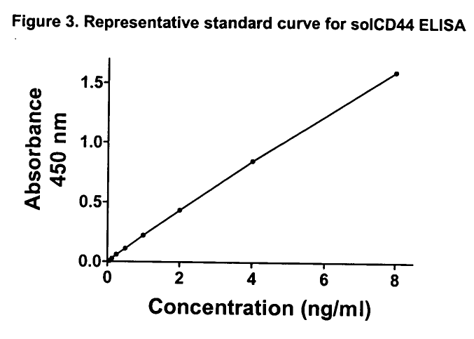

The standard curve was generated using cubic spline curve fit. Standard curves were run in duplicate on each plate. Coefficient of determination ranged from 0.98-0.99 for all of the standard curves. A representative curve is shown in FIG. 3.

Precision

The precision of an assay is defined by the agreement between replicate measures (88). Samples (73 HNSCC and 54 control specimens) were repeated in duplicate at full concentration, 1:2 and 1:4 dilutions. The average coefficient of variation for the resulting 381 duplicate measurements was 4.5 %.

Samples were run on a total of 17 ELISA plates. Since a reference standard is not available, we prepared the positive control sample containing 59 ng/ml of recombinant solCD44 in synthetic saliva diluted 1:5 in normal saline. This positive control was run in duplicate on each plate to assess differences between plates. Average coefficient of variation for duplicate readings of the positive control was 3.6%. Coefficient of variation between plates was 9.7%.

Sensitivity

Analytical sensitivity is defined as the lowest concentration detected that is significantly different than zero (158). Mean absorbance of our blanks run on 17 different plates was 0.02±0.015. We defined 3 standard deviations as significantly different and calculated the corresponding concentration from a representative standard curve. Using this method the analytical sensitivity of the test is 0.091 ng/ml.

Specificity

Specificity of the CD44 antibody is described in detail in the Bender MedSystems Manual. They detected no cross reactivity between this test and TNF-α, TNF-β, TNF-R, IFN-α2c, INF-γ, IL-8 annexin, sELAM-1, s1-selectin, s1CAM1, or HER-2.

As defined herein, a matrix effect is an interference in the solCD44 test resulting from a property of or factor within the medium, i.e. saliva. Ideally, when a saliva sample is serially diluted and tested, resulting absorbances, when plotted against concentration, should yield a line with the same slope as the standard curve. Any deviation from that line signifies a matrix effect. To test for matrix effects, samples were run at three serial dilutions, results were plotted, slopes calculated and compared to the slope in the same region of the standard curve. Since our standard curve was not perfectly linear, this method is an approximation. Therefore we set an arbitrary cut-off at 30% of the standard curve. Any sample yielding a slope that deviated more than 30% from the standard slope was considered to have a matrix effect. One of the HNSCC samples lacked serial dilutions and matrix effect was not examined. Seven of the 73 HNSCC and none of the control specimens had solCD44 levels above the highest standard precluding analysis of matrix effects. Twelve of the 54 control specimens and two of the tumor specimens had very low solCD44 levels (below 0.5 ng/ml) where the standard curve was less linear and results more uncertain. Excluding these 14 samples, 21% of the tumor samples and 24% of the control samples showed evidence of a matrix effect. Chi-square analysis confirmed that these two groups were not different regarding the presence of matrix effect (p=0.85)

Sample temperature appears to contribute to this matrix effect. Three samples one patient control, one tumor, and our synthetic saliva positive control were tested in duplicate serial dilutions under two conditions. In the first condition, samples were thawed on ice and kept on ice until loaded on the plate. In the second condition the samples were brought to room temperature for 45 minutes and left at room temperature until loaded on the plate. Levels were significantly increased (15-20%) in the room temperature condition compared to the condition on ice for the tumor sample (p<0.005), the patient control (p<0.01) and the synthetic control (p<0.01). The patient control sample had a matrix effect on ice on the original run and in this experiment. When the sample was brought to room temperature, analysis of serial dilutions showed resolution of the matrix effect. Because of these findings, assays for the remainder of our assessment were performed after bringing the specimens to room temperature for 45 minutes. We repeated solCD44 levels on serial dilutions of 3 normal and 3 tumor samples that had matrix effects this time bringing all samples to room temperature for 45 minutes. We chose samples with low, medium, and high concentrations of solCD44 on the original run. All of the matrix effects resolved using the new method, except in the two samples with low solCD44.

Recovery

Identical standard curves were prepared in synthetic saliva and 4 different human saliva specimens. Slopes were calculated using linear regression. We compared slopes between the standard curve in synthetic saliva and the standard curves in human saliva samples as a measure of recovery. Results are shown in Table 7. In this table recovery %=slope of sample curve/slope of standard curve X 100.

TABLE 7
Recovery

| Sample | Recovery % |
|---|---|
| 1 | 94% |
| 2 | 61% |
| 3 | 95% |
| 4 | 77% |

Freeze-Thaw Cycles

Three samples were aliquotted into five tubes and stored at −80°. SolCD44 levels were tested for each aliquot of a sample and coefficient of variation between aliquots of the same sample was determined. Aliquots were then taken through one to five freeze-thaw cycles. SolCD44 levels were measured for each sample at one, three and five freeze-thaw cycles and the coefficient of variation between freeze-thaw cycles of the same sample was measured. Results are shown below in Table 8. Coefficients of variation between aliquots and between freeze-thaw cycles were similar and below 20% in all cases indicating that neither freeze-thaw cycles nor dividing into aliquots significantly affected results.

TABLE 8
Freeze-thaw cycles

| Sample | Freeze-thaw % CV | Aliquot % CV |
|---|---|---|
| 1 | 2.4 | 6.1 |
| 2 | 14.1 | 15.7 |
| 3 | 14.0 | 17.5 |

Stability

Three saliva samples were aliquoted and stored at 4° C. and room temperature for 24 hours. Levels of solCD44 did not decrease significantly after storage at room temperature or 4° C. compared to storage at −80° C.

Summary of Results

Our results suggest that early disease can be detected with the solCD44 test. Currently most HNSCC is diagnosed in stage III or IV when treatment is less effective and cure rates are below 40% (2,5-7). It is estimated that if HNSCC were diagnosed in early stages cure rates could exceed 80% (7). A single modality treatment, surgery or radiation, is effective in treating most early stage lesions (4,6). Not only is prognosis improved with early detection, but morbidity is also reduced (fewer post-treatment speech, swallowing and breathing problems than those with advanced stage disease) (70). Because the tumors are smaller, there is also less disfigurement (70).

SolCD44 levels were elevated in HNSCC patients compared to the controls (p<0.001). The test detected 70-79% of invasive HNSCC using preliminary cut-off points. SolCD44 levels did not vary significantly with tumor size, stage, recurrence, history of radiation treatment, or tobacco and alcohol risk factors. A 65-75 kDa band, corresponding to solCD44, was detected in all of the HNSCC cell line CM and saliva while normal samples showed a fainter band or were undetectable. Tobacco use, alcohol consumption, race, age, socioeconomic status, gender and general oral health do not appear to effect levels of solCD44 in saliva.

Salivary solCD44 ELISA appears to effectively detect HNSCC at all stages. Since early detection of HNSCC results in significantly improved survival, the salivary solCD44 test should be an effective HNSCC screening tool.

References are cited below and are hereby incorporated by reference.
1. Muir C and Weiland L. Upper aerodigestive tract cancers. Cancer Suppl 1995; 75:147-53.
2. Lingen M, Sturgis E M and Kies M S. Squamous cell carcinoma of the head and neck in nonsmokers: clinical and biologic characteristics and implications for management. Curr Opin Oncol. 2001; 13:176-82.
Jesse R H and Sugarbaker E V. Squamous cell carcinoma of the oropharynx: why we fail. Am J Surg 1976; 132:435-438.
3. Jefferies S, Eeles R, Goldgar D, A'Hern R, Henk J M, Gore M, MPT collaborators: Rhys-Evans P, Archer D, Bishop K, Murkin A, Solomon E, Hodgsun S, O'Connell M, Hibbert J, Easton D and Foulkes W. The role of genetic factors in predisposition to squamous cell cancer of the head and neck. Br J Cancer 1999; 79:865-7.
4. Forastiere A, Koch W, Trotti A, Sidransky D. Head and neck cancer. N Engl J Med 2001; 345:1890-1900.
5. Al-Sarraf M. Treatment of locally advanced head and neck cancer: historical and critical review. Cancer Control 2002; 9:387-99
6. Vokes E E, Weichselbaum R R, Lippman S M and Hong W K. Head and neck cancer. N Engl J Med. 1993; 328:184-94.
7. O'Hara J and Bradley P. Head and neck cancer: a screening strategy. Clin Otolaryngol 2002; 27:133-4.
8. Cancer Facts and Figures—2002. Atlanta:American Cancer Society, 2002.
9. Jones A S, Beasley N, Houghton D and Husband D J. The effects of age on survival and other parameters in squamous cell carcinoma of the oral cavity, pharynx and larynx. Clin Otolaryngol 1998; 23:51-6.
10. American Joint Committee on Cancer Staging, 5th ed. Philadelphia: Lippincott Williams and Wilkins, 1998.
11. Neville B W and Day T A. Oral cancer and precancerous lesions. CA Cancer J Clin 2002; 52:195-215.
12. Dolan R W, Vaughan C W and Fuleihan N. Symptoms in early head and neck cancer: an inadequate indicator. Otolaryngol Head Neck Surg 1998; 119:463-7.
13. National Cancer Institute/US. Oral cancers: research report. US Department of Health and Human Services, Public Health Service, National Institute of Health 1991; NIH publication no. 92-2876.
14. Weinberg M A and Estefan D J. Assessing oral malignancies. Am Fam Physician. 2002; 65:1379-84.
15. Spafford M F, Koch W M, Reed A L, Califano J A, Xu L H, Eisenberger C F, Yip L, Leong P L, Wu L, Liu S X, Jeronimo C, Westra W H and Sidransky D. Detection of head and neck squamous cell carcinoma among exfoliated oral mucosal cells by microsatellite analysis. Clin Cancer Res 2001; 7:607-12.
16. Reyes C M, Allen B A, Terdiman J P and Wilson L S. Comparison of selection strategies for genetic testing of patients with hereditary nonpolyposis colorectal carcinoma. Cancer 2002; 95:1848-56.
17. Ellison L, Cheli C D, Bright S, Veltri R W and Partin A W. Cost-benefit analysis of total, free/total, and complexed prostate-specific antigen for prostate cancer screening. Urology 2002; 60(Suppl 4A):42-6.
18. Lokeshwar V B and Soloway M S. Current bladder tumor tests: does their projected utility fulfill clinical necessity? J Urol 2001; 165:1067-77.
19. Franzmann E J, Schroeder G L, Goodwin W J, Weed D T, Fisher P and Lokeshwar V B. Expression of tumor markers hyaluronic acid and hyaluronidas (HYAL1) in head and neck tumors. Int J. Cancer 2003; 106:438-445.
20. Lokeshwar V B, Iida N, and Bourguignon L Y W. The cell adhesion molecule, GP116, is a new CD44 variant (ex14/v10) involved in hyaluronic acid binding and endothelial cell proliferation. J Biol Chem 1996; 271:23853-64.
21. Brown T A, Bouchard T, St. John T , Wayner E and Carter W G. Human keratinocytes express a new CD44 core protein (CD44E) as a heparin-sulfate intrinsic membrane proteoglycan with additional exons. J Cell Biol 1991; 113:207-21.
22. Omary M B, Trowbridge I S, Letarte M, Kagnoff M F and Isacke C M. Structural heterogeneity of human Pgp-1 and its relationship with p85. Immunogenetics 1988; 27:460-4.
23. Trowbridge I S, Lesley J, Schulte R, Hyman R and Trotter J. Biochemical characterization and cellular distribution of a polymorphic, murine cell-surface glycoprotein expressed on lymphoid tissues. Immunogenetics 1982; 15:299-312.
24. Bourguignon L Y W, lida N, Welsh C F, Zhu D, Krongrad A and Pasquale D. Involvement of CD44 and its variant isoforms in membrane-cytoskeleton interaction, cell adhesion, and tumor metastasis. J Neurooncol 1995; 26:201-8.
25. Screaton G R, Bell M V, Jackson D G, Cornelis F B, Gerth V and Bell J I. Genomic structure of DNA encoding the lymphocyte homing receptor CD44 reveals at least 12 alternatively spliced exons. Proc Natl Acad Sci USA 1992; 89:12160-4.
26. Naor D, Sionov R V and Ish-Shalom D. CD44: Structure, function, and association with the malignant process. Adv Cancer Res. 1997; 71:241-319.
27. lida N and Bourguignon L Y W. New CD44 splice variants associated with human breast cancers. J Cell Physiol 1995; 162:127-33.
28. Franzmann E J, Weed D T, Civantos F J, Goodwin W J and Bourguignon L Y W. A novel CD44v3 isoform is involved in head and neck squamous cell carcinoma progression. Otolaryngol Head Neck Surg 2001; 124:426-32.
29. Bourguignon L Y W, Lokeshwar V B, He J, Chen X and Bourguignon G J. A CD44-like endothelial cell transmembrane glycoprotein (GP116) interacts with extracellular matrix and ankyrin. Mol Cell Biol 1992; 12:4464-71.
30. Kajita M, Itoh Y, Chiba T, Mori H, Okada A, Kinoh H and Seiki M. Membrane-type 1 matrix metalloproteinase cleaves CD44 and promotes cell migration. J. Cell Biol 2001; 153:893-904.
31. Guo Y J, Liu G, Wang X, Jin D, Wu M, Ma J and Sy M S. Potential use of soluble CD44 in serum as an indicator of tumor burden and metastasis in patients with gastric or colon cancer. Cancer Res 1994; 54:422-6.
32. Martin S, Jansen F, Bokelmann J and Kolb H. Soluble CD44 splice variants in metastasizing human breast cancer. Int J Cancer 1997; 74:443-445.
33. Ristamaki R, Joensuu H, Lappalainen K, Teerenhovi L and Jalkanen S. Elevated serum CD44 level is associated with unfavorable outcome in non-Hodgkin's lymphoma. Blood 1997; 90:4039-4045.
34. Yamane N, Tsujitani S, Makino M, Maeta M and Kaibara N. Soluble CD44 variant 6 as a prognostic indicator in patients with colorectal cancer. Oncology 1999; 56:232-8.
35. Scott D A, Stapleton J A, Palmer R M, Wilson R F, Sutherland G, Coward P Y, Gustavsson G, Odell E W and Poston R N. Plasma concentrations of reputed tumor-associated soluble CD44 isoforms (v5 and v6) in smokers are dose related and decline on smoking cessation. Cancer Epidemiol Biomarkers Prev 2000; 9:1211-4.
36. Van Hal N L W, Van Dongen G A M S, Ten Brink C B M, Heider K H, Rech-Weichselbraun I, Snow G B and Brakenhoff R H. Evaluation of soluble CD44v6 as a potential serum marker for head and neck squamous cell carcinoma. Clin Cancer Res 1999; 5:3534-41.
37. Dall P, Heider K H, Sinn H P, Skroch-Angel P, Adolf G, Kaufmann M, Herrlich P and Ponta H. Comparison of immunohistochemistry and RT-PCR for detection of CD44v-expression, a new prognostic factor in human breast cancer. Int J Cancer 1995:60:471-77.
38. Rodriguez C, Monges G, Rouanet P, Dutrillaux B, Lefrancois D and Theilett C.CD44 expression patterns in breast and colon tumors: A PCR-based study of splice variants. Int J Cancer 1995; 64:347-54.
39. Kaufmann M, Heider K H, Sinn H P, Von Minckwitz G, Ponta H and Herrlich P. CD44 variant exon epitopes in primary breast cancer and length of survival. Lancet 1995; 345:615-9.
40. Knudson W. The role of CD44 as a cell surface hyaluronan receptor during tumor invasion of connective tissue. Front Biosci. 1998; 3:604-15.
41. Holzer G, Kittl E, Pfandelsteiner T, Trieb K and Kotz R. Concentration of soluble CD44 Standard and Soluble CD44v5 in the serum of patients with malignant bone tumors. Med Pediatr Oncol. 2003; 40:64-5.
42. Sheen-Chen S, Chen W, Eng H, Sheen C, Chou F and Cheng Y. Evaluation of the prognostic value of serum soluble CD44 in patients with breast cancer. Cancer Investigation 1999; 17:581-5.
43. Masson D, Denis M G, Denis M, Blanchard D, Loirat M J, Cassagnau E and Lustenberger P. Soluble CD44:quantification and molecular repartition in plasm of patients with colorectal cancer. Br J Cancer. 1999; 80:1995-2000.
44. Niitsu N and Iijima K. High serum soluble CD44 is correlated with a poor outcome of aggressive non-Hodgkin's lymphoma. Leukemia Res. 2002; 26:241-8.
45. Molica S, Vitelli G, Levato D, Giannarelli D and Gandolfo G M. Elevated serum levels of soluble CD44 can identify a subgroup of patients with early B-cell chronic lymphocytic leukemia who are at high risk for disease progression. Cancer. 2001; 92:713-9.
46. Girja K, Sundharam B, Krishnan P and Devi C. Biochemical changes of saliva in tobacco chewers, tobacco smokers, alcohol consumers, leukoplakia and oral cancer patients. IJDR 2002; 13:102-7.
47. Etzioni R, Urban N, Ramsey S, McIntosh M, Schwartz S, Reid B, Radich J, Anderson G and Hartwell L. The case for early detection. Nature Rev Cancer 2003; 3:243-52.
48. Ries L A G, Eisner M P, Kosary C L, Hankey B F, Miller B A, Clegg L, Mariotto A, Fay M P, Feuer E J, Edwards B K (eds). SEER Cancer Statistics Review, 1975-2000, National Cancer Institute. Bethesda, Md., http://seer.cancer.gov/csr/1975_{—}2000, 2003.
49. Zakrzeweska J M. Oral cancer. BMJ 1999; 318:1051-54.
50. Koch W M and McQuone S. Clinical and molecular aspects of squamous cell carcinoma of the head and neck in the nonsmoker and nondrinker. Curr Opin Oncol 1997; 9:257-61.
51. Gillison M L, Koch W M and Shah K V. Human papillomavirus in head and neck squamous cell carcinoma: are some head and neck cancers a sexually transmitted disease? Curr Opin Oncol 1999; 11:191-99.
52. Blot W J. McLaughlin J K, Winn D M, Austin D F, Greenberg R S, Preston-Martin S, Bernstein L, Schoenberg J B, Stemhagen A and Fraumeni J F. Smoking and drinking in relation to oral and pharyngeal cancer. Cancer Res. 1988; 48:3282-7.
53.Winn D M, Blot W J, Shy C M, Pickle L W, Toledo A and Fraumeni J F. Snuff dipping and oral cancer among women in the Southern United States. N Engl J Med 1981; 304:745-9.
54. Garrote L F, Herrero R, Reyes R M O, Vaccarella S, Anta J L, Ferbeye L, Munoz N and Franceschi S. Risk factors for cancer of the oral cavity and oro-pharynx in Cuba. Br J Cancer 2001; 85:46-54.
55. Shah J P, Cendon R A, Farr H W, and Strong E W. Carcinoma of the oral cavity. Am J Surg 1976; 132:504-507.
56. Goodwin W J. Salvage surgery for patients with recurrent squamous cell carcinoma of the upper aerodigestive tract: when do the ends justify the means? Laryngoscope 2000; 110:S1-18.
57. Warnakulasuriya K A A S and Johnson N W. Sensitivity and specificity of Orascan toluidine blue mouthrinse in the detection of oral cancer and precancer. J Oral pathol Med 1996; 25:97-103.
58. Warnakulasuriya S. Lack of markers to predict potential of oral precancer (editorial). J Pathol 2000; 190:407-9.
59. Gallo A, de Vincentiis M, Rocca C D. Moi R, Simonelli M, Minni A, and Shaha A R. Evolution of precancerous laryngeal lesions: a clinicopathologic study with long-term follow-up on 259 patients. Head Neck 2001; 23:42-7.
60. Subdo J, Kildal W, Risberg B. Koppang H S, Danielsen H E and Reith A. DNA content as a prognostic marker in patients with oral leukoplakia. N Engl J Med 2001; 344: 1270-8.
61. Werkmeister R, Brandt B and Joos U. Aberrations of erbB-1 and erbB-2 oncogenes in non-dysplastic leukoplakias or the oral cavity. Br J Oral Maxillofac Surg 1999; 37:477-80.
62. Hamidi S, Salo T, Kainulainen T, Epstein J, Lerner K and Larjava H. Expression of alpha (V) beta 6 integrin in oral leukoplakia. Br J Cancer 2000; 82: 1433-40.
63. Hu Y C, Sidransky D and Ahrendt S A. Molecular detection approaches for smoking associated tumors. Oncogene 2002; 21:7289-97.
64. Sidransky D. Emerging molecular markers of cancer. Nature Rev Cancer 2002; 2:210-19.
65. Boyle J O, Mao L, Brennan J A, Koch W M, Eisele D W, Saunders J R and Sidransky D. Gene mutations in saliva as molecular markers for head and neck squamous cell carcinomas. Am J Surg 1994; 168:429-32.
66. Rosas S L B, Koch W, da Gloria da Costa Carvalho M, Wu L, Califano J, Westra W, Jen J and Sidransky D. Promoter hypermethylation patterns of p16.0⁶-methylguanine-DNA-methyltransferase, and death-associated protein kinase in tumors and saliva of head and neck cancer patients. Cancer Res 2001; 61:939-42.
67. Fliss M S, Usadel H, Caballero O L, Wu L, Buta M R, Eleff S M, Jen J and Sidransky D. Facile detection of mitochondrial DNA mutations in tumors and bodily fluids. Science 2000; 287:2017-9.
68. Nawroz H, Koch W, Anker P, Stroun M and Sidransky D. Microsatellite alterations in serum DNA of head and neck cancer patients. Nat Med 1996; 2:1035-7.
69. Sanchez-Cespedes M, Esteller M, Wu L, Nawroz-Danish H, Yoo G H, Koch W M. Jen J, Herman J G and Sidransky D. Gene promoter hypermethylation in tumors and serum of head and neck cancer patients. Cancer Res 2000; 60:892-5.
70. Sanderson R J and Ironside J A D. Squamous cell carcinomas of the head and neck. BMJ. 2002; 325:822-7.
71. The International Prostate Screening Trial Evaluation Group. Rationale for randomized trials of prostate cancer screening. Eur J Cancer. 1999; 35:262-71.
72. Nanda, K McCrory D C, Myers E R, Bastian L A, Hasselblad V, Hickey J D and Matchar D B. Accuracy of the Papanicolaou Test in screening for and follow-up of cervical cytologic abnormalities: a systemic review. Ann Intern med 2000; 132:810-9.
73. Scully C, Sudbo J and Speight P M. Progress in determining the malignant potential of oral lesions. J Oral Pathol Med 2003; 32:251-6.
74. St. John M A R, Li Y, Zhou X, Denny P, Ho C, Montemango C, Shi W, Qi F, Wu B, Sinha U, Jordan R, Wolinsky L, Park N, Liu H, Abemayor E and Wong D T W. Interleukin 6 and interleukin 8 as potential biomarkers for oral cavity and oropharyngeal squamous cell carcinoma. Arch Otolaryngol Head Neck Surg 2004; 130:929-935.
75. Li Y, St. John M A R, Zhou X, Kim Y, Sinha U, Jordan R C K, Eisele D, Abemayor E, Elashoff D, Park N and Wong D T. Salivary transcriptome diagnostics for oral cancer detection. Clin Cancer Res 2004; 10:8442-50.
76. Califano J, Ahrendt S A, Meininger G, Westra W H, Koch W M and Sydransky D. Detection of telomerase activity in oral rinses from head and neck squamous cell carcinoma patients. Cancer Res 1996; 56:5720-2.
77. loachim E, Assimakopoulos D, Goussia A C, Peschos D, Skevas A and Agnantis N J. Glycoprotein CD44 expression in benign, premalignant and malignant epithelial lesions of the larynx: an immunohistochemical study including correlation with Rb, p53, Ki-67 and PCNA Histol Histopathol. 1999; 14:1113-8.
78. Reszec J, Sulkowska M, Famulski W, Guzinihska-ustymowisc K and Sulkowski S. The expression of tumorigenesis markers in oral papilloma. Pol J Path. 2002; 53:195-200.
79. Maula S, Luukkaa M, Grenman R, Jackson D, Jalkanen S and Ristamaki R. Intratumoral lymphatics are essential for the metastatic spread and prognosis in squamous cell carcinomas of the head and neck region. Cancer Res 2003; 1920-6.
80. Kim M M and Califano, J A. Molecular pathology of head and neck cancer. Int J Cancer 2004; 112:545-553.
81. Streckfus C F and Bigler L R. Saliva as a diagnositic fluid. Oral Diseases 2002; 8:69-76.
82. Humphrey S P and Williamson R T. A review of saliva: normal composition, flow and function. J Prosthet Dent 2001; 85:162-9.
83. Pedersen A M, Bardow A, Jensen S B and Nauntofite B. Saliva and gastrointestinal functions of taste, mastication, swallowing and digestion. Oral Diseases 2002; 8:117-29.
84. MacKay A, Moehl T and Slotar S. The effect of body hydration state on oral fluids. Diastema 1986; 14:5-7,9.
85. Lokeshwar V B and Bourguignon L Y W. Post-translational protein modification and expression of ankyrin-binding site(s) in GP85 (Pgp-1/CD44) and its biosynthetic precursors during T-lymphoma membrane biosynthesis. J Biol Chem 1991; 266:17983-9.
86. Jackson D G, Bell J I, Dickinson R, Timans J, Shields J and Whittle N. Proteoglycan forms of the lymphocyte homing receptor CD44 are alternatively spliced variants containing the v3 exon. J Cell Biol 1995; 128:673-85.
87. Assimakopoulos D, Kolettas E, Patrikakos G, Evangelou A. The role of CD44 in the development and prognosis of head and neck squamous cell carcinomas. Histol Histopathol. 2002 17:1269-81.
88. Sweep F C G J, Fritsche H A, Gion M, Klee G G and Schmitt M. Considerations on development, validation, application, and quality control in immuno(metric) biomarker assays in clinical cancer research: An EORTC-NCI working group report. Int J Oncol 2003; 23:1715-26.
89. Nakamura H, Suennaga N, Taniwaki K, et al. Constitutive and induced CD44 shedding by ADAM-like proteases and membrane-type 1 matrix metalloproteinase. Cancer Res 2004; 64:876-82.

1. A method of diagnosing head and neck squamous cell carcinoma (HNSCC) in a subject, comprising measuring soluble CD44 (solCD44) in a biological sample obtained from said subject, wherein an elevated level of solCD44 in the biological sample compared to a baseline from a normal population of subjects is indicative of the possible presence of HNSCC.

2. The method of claim 1 wherein the subject is a human.

3. The method of claim 1, wherein the biological sample comprises a bodily fluid.

4. The method of claim 3, wherein the bodily fluid is saliva.

5. The method of claim 1 wherein the HNSCC is early stage.

6. A method of predicting the course of HNSCC in a subject, comprising measuring solCD44 in a biological sample obtained from said subject, wherein the degree of elevation of the level of solCD44 in the biological sample compared to a baseline from a normal population of subjects is indicative of the severity of disease.

7. The method of claim 6 wherein the subject is a human.

8. The method of claim 6, wherein the biological sample comprises a bodily fluid.

9. The method of claim 8, wherein the bodily fluid is saliva.

10. The method of claim 6 wherein the HNSCC is early stage.

11. A method of predicting the success of treatment of HNSCC in a subject, comprising measuring solCD44 in a biological sample obtained from said subject, wherein the degree of elevation of the level of solCD44 in the biological sample compared to a baseline from a normal population of subjects is predictive of outcome, with a low elevation level being more favorable than a high elevation level.

12. The method of claim 11 wherein the subject is a human.

13. The method of claim 11, wherein the biological sample comprises a bodily fluid.

14. The method of claim 13, wherein the bodily fluid is saliva.

15. The method of claim 11 wherein the HNSCC is early stage.

16. A method of determining the effectiveness of treatment of HNSCC in a subject, comprising measuring solCD44 in a biological sample obtained from said subject, wherein a decrease in the level of solCD44 in the biological sample compared to a previously measured level is indicative of effective treatment.

17. The method of claim 16 wherein the subject is a human.

18. The method of claim 16, wherein the biological sample comprises a bodily fluid.

19. The method of claim 18, wherein the bodily fluid is saliva.

20. The method of claim 16 wherein the HNSCC is early stage.

21. A method of diagnosing head and neck squamous cell carcinoma (HNSCC) in a subject, comprising measuring soluble CD44 (solCD44) in saliva obtained from said subject, wherein an elevated level of solCD44 in the saliva of said subject compared to a baseline of CD44 (solCD44) in saliva of a normal population of subjects is indicative of the possible presence of HNSCC.

22. A method of predicting the course of HNSCC in a subject, comprising measuring solCD44 in a biological sample obtained from said subject, wherein the degree of elevation or depression of the level of solCD44 in the biological sample compared to a baseline from an earlier sample from said subject is indicative of the severity of disease, with greater elevation indicating a poorer prognosis, and greater depression indicating a favorable prognosis.

23. A method of predicting recurrence of HNSCC in a subject, comprising measuring solCD44 in a biological sample obtained from said subject, wherein an increase of solCD44 in the biological sample compared to a baseline from an earlier sample from said subject is indicative of possible recurrence of the disease in the subject.
